(19) United States
(12) Patent Application Publication
(10) Pub. No.: US 2009/0126743 A1
(43) Pub. Date: May 21, 2009

(54) Apparatus and method of a locality specific pre-hospital pediatric medical treatment

(76) Inventor: William P. Wingert, Santa Cruz, CA (US)

Correspondence Address: MURABITO HAO & BARNES LLP, Third Floor, Two North Market Street, San Jose, CA, 95113, US

(21) Appl. No.: 11/986,582

(22) Filed: Nov. 21, 2007

(51) Int. Cl. A61B 19/00; G01B 3/10

(52) U.S. Cl. 128/898; 33/755

(57) ABSTRACT

Methods and an apparatus of locality specific pre-hospital pediatric emergency medical treatment are disclosed. In one embodiment, the method includes preparing an emergency manual conforming to a guideline pertaining to the locality specific pre-hospital pediatric medical treatment. The method further includes determining items according to the emergency manual and furnishing a portable container with the items.

### FIELD OF TECHNOLOGY

This disclosure relates generally to technical fields of emergency medicine, in one embodiment, to pre-hospital pediatric emergency response methods and apparatus.

### BACKGROUND

A color coded tape (e.g., Broselow Tape of U.S. Pat. No. 4,713,888 and U.S. Pat. No. 4,823,469) is used to determine the physical treatment (e.g., the dosage, the equipment, etc.) and/or procedure of a patient in a pre-hospital and/or hospital environment. The color coded tape is first used to approximate the weight of the patient, and the physical treatment and/or procedure of the patient is administered based on the category of the patient determined by the weight.

In addition to the color coded tape, a therapeutic apparatus (e.g., U.S. Pat. No. 5,010,656 and U.S. Pat. No. 4,926,885) includes a dispenser with a series of colored drawers matching the color coding scheme of the color coded tape. Each of the colored drawers contains a medical or surgical apparatus (e.g., an endotracheal tube) or pre-packed drugs which are required for the physical treatment and/or procedure of the patient with his or her weight matching the color in one of the drawers.

However, the therapeutic apparatus may not be ideal for the pre-hospital environment and is not customized for the pre-hospital environment. For example, on one side of the color coded tape (e.g., the Broselow Tape) is often crowded with too much information which includes the details of the physical treatment and/or procedure not permitted by pre-hospital care providers, thus making it difficult for a caregiver to follow the guidelines in an emergency setting. In addition, the dispenser may be too big, cumbersome and/or too heavy to carry around to treat the patient under the pre-hospital environment (e.g., and/or to fit into an ambulance, a fire truck, etc.).

Moreover, the therapeutic apparatus is not often customized to meet the guideline set out by a local government (e.g., a county, a city, a municipality, etc.) since it is often based on the federal guidelines. With the lack of a clear local guideline, emergency workers tend to rely more on their instincts under the duress of the pre-hospital environment. As a result, drug dosages and/or medical equipment administered by the emergency workers may not be ideal for some patients, and their delivery of the medical service may be hampered by unclear instructions, thus increasing the potential for liability. This becomes especially problematic when the patient is a little child or an infant where their weight class requires special equipment and dosages that need to be located, measured, and/or administered promptly.

### SUMMARY

Accordingly, what is needed is more efficient manner of administering pre-hospital pediatric emergency care. What is further needed is equipping a care provider with equipment, medications, and instructions based on a locality specific pediatric emergency protocol so that the care provider can promptly attend to a patient with utmost care and remain safely within his or her scope of practice. What is further needed is an effective manner of treating children and small adults in a pre-hospital environment potentially involving emergency responses. Embodiments of the present invention provide these advantages.

One embodiment of the present invention pertains to an apparatus for locality specific pre-hospital pediatric medical treatment. The apparatus includes a measuring tape divided into multiple sections with each section representing a category of a patient and an emergency manual based on the locality specific pre-hospital pediatric medical treatment listing information for treating the patient. The apparatus further includes one or more pouches containing items mandated by the emergency manual. In this embodiment, the pouches correspond to different weight classes of patients divided into the multiple sections.

In another example embodiment, a method includes measuring a patient to determine a category of the patient and carrying out one or more instructions listed in an emergency treatment manual to treat the patient where the emergency treatment manual is locality specific in its protocol.

As illustrated in the detailed description, other embodiments also pertain to methods and apparatus that provide an enhanced pre-hospital emergency response, and in particular, improving the accuracy and speed in the delivery of the pre-hospital pediatric emergency care. Through adapting to the locality specific pre-hospital pediatric medical treatment, the embodiments aim to provide more efficient medical service to care for children.

### BRIEF DESCRIPTION OF THE DRAWINGS

Example embodiments are illustrated by way of example and not limitation in the figures of the accompanying drawings, in which like references indicate similar elements and in which:

FIG. 1A is an exemplary diagram of a color coded tape, according to one embodiment.

FIG. 1B is an exemplary view of a category table matching a color with a weight, according to one embodiment.

FIG. 2 is an exemplary view of a patient measured by the color coded tape of FIG. 1A, according to one embodiment.

FIG. 3 is an exemplary view of a carry-on with multiple compartments, according to one embodiment.

FIG. 4 is an exemplary view of the main compartment of the carry-on of FIG. 3 having multiple pouches in different colors, according to one embodiment.

FIG. 5A is an exemplary view of an emergency manual listing symptoms and a packing list, according to one embodiment.

FIG. 5B is an exemplary page view of the emergency manual of FIG. 5A listing instructions, according to one embodiment.

FIG. 5C is an exemplary page view of the emergency manual of FIG. 5A listing instructions, according to one embodiment.

FIG. 6 is an exemplary process flow chart of furnishing a portable container based on a locality specific pre-hospital pediatric medical treatment guideline, according to one embodiment.

FIG. 7 is an exemplary process flow chart of administering a locality specific pre-hospital pediatric medical treatment, according to one embodiment.

FIG. 8 is an exemplary view of a pre-hospital emergency care agency serving emergency needs of three different municipalities by methods and tools described in FIGS. 1A through 7, according to one embodiment.

Other features of the present embodiments will be apparent from the description that follows.

### DETAILED DESCRIPTION

Reference will now be made in detail to the preferred embodiments of the present invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and circuits have not been described in detail as not to unnecessarily obscure aspects of the present invention.

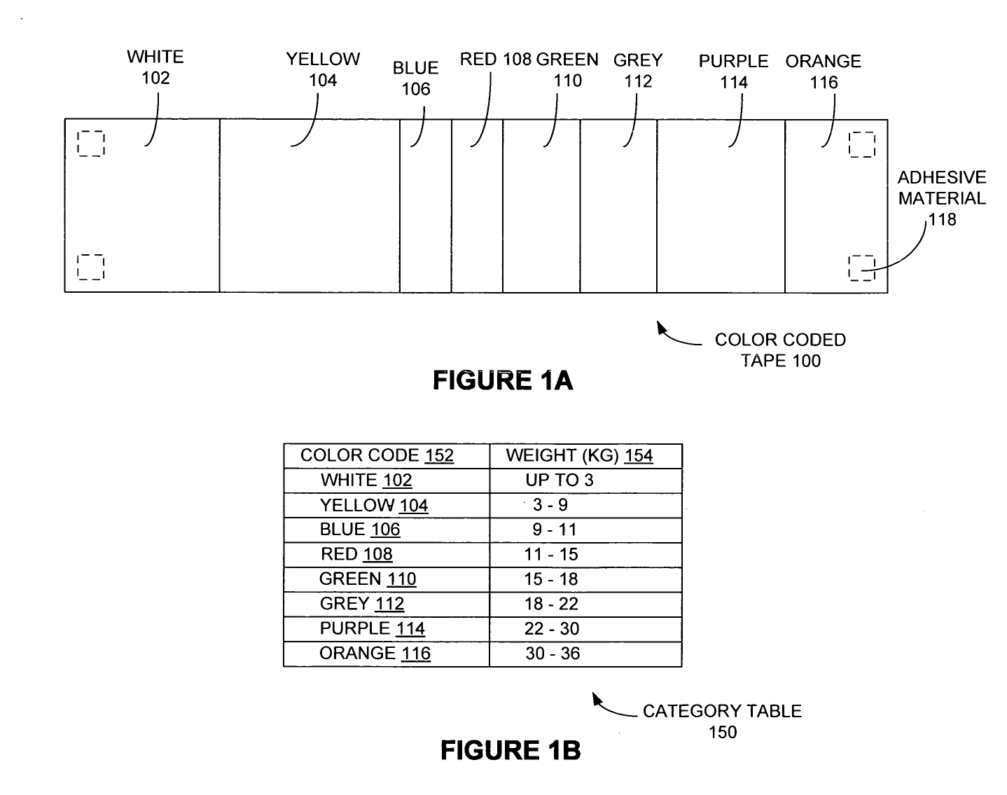

FIG. 1A is an exemplary diagram of a color coded tape 100, according to one embodiment. FIG. 1A illustrates a measuring tape with a color code scheme. In one example embodiment, the measuring tape divided into multiple sections for aligning with a patient (e.g., on the ground) is used to measure a size (e.g., a weight and/or a height) of the patient to assign the patient to one of a category associated with a pre-hospital pediatric medial treatment.

In another example embodiment, the measuring tape is made of a material (e.g., plastic) such that the measuring tape does not curl up to hinder the measurement of the patient. Also, an adhesive material 118 (e.g., Velcro) or heavy weights applied to edges of the measuring tape may be used to prevent the curling up of the measuring tape during the measurement. In yet another example embodiment, the measuring tape is the color coded tape 100 comprising multiple sections of a white 102, a yellow 104, a blue 106, a green 110, a grey 112, a purple 114, and an orange 116 (e.g., with each color corresponding to a weight class). The measuring tape may measure an approximate weight for stature between 0 kilograms and 36 kilograms.

FIG. 1B is an exemplary view of a category table 150 matching a color with a weight 154 of a patient (e.g., a child patient), according to one embodiment. In the treatment category table 150 (e.g., which may be based on the nationwide statistical survey, such as NHANES III data, of matching the height of the child patient with his/her weight), a color code 152 is matched with the weight 154 (e.g., approximate) of the patient. In one example embodiment, the white 102 is matched with the weight 154 between 0 to 3 kilograms, the yellow 104 with the weight 154 between 3 and 9 kilograms, the blue 106 with the weight 154 between 9 and 11 kilograms, the red 108 with the weight 154 between 11 and 15 kilograms, the green 110 with the weight 154 between 15 and 18 kilograms, the grey 112 with the weight 154 between 18 and 22 kilograms, the purple 114 with the weight 154 between 22 and 30 kilograms, and the orange 116 with the weight 154 between 30 and 36 kilograms.

The division of the weight groups may be predetermined based on the size of following equipment: Oropharyngeal airway device, endotracheal tube, laryngoscope blade, tube stylet, bag value mask device, suction catheter, and/or IV angiocatheter. In other words, for each weight category, a different size of the equipment may be required. Additionally, the relationship between the weight of the patient and his or her drug dose may play an important role in generating the category table 150. That is, dosages may be dependent on the weight class. The color code 152 for a particular patient based on the category table 150 (e.g., which may be mandated by a local government protocol) may enable a care provider (e.g., a paramedic, an emergency care worker, etc.) of the patient to quickly attend to the patient through locating a relevant care package (e.g., which may contain a medical equipment, a mediation, and/or an emergency treatment manual) that is customized for the patient and for the locality where the emergency is taking place. It is appreciated that paramedic practice dealing with such an emergency is often governed by very specific guidelines unique to each locality.

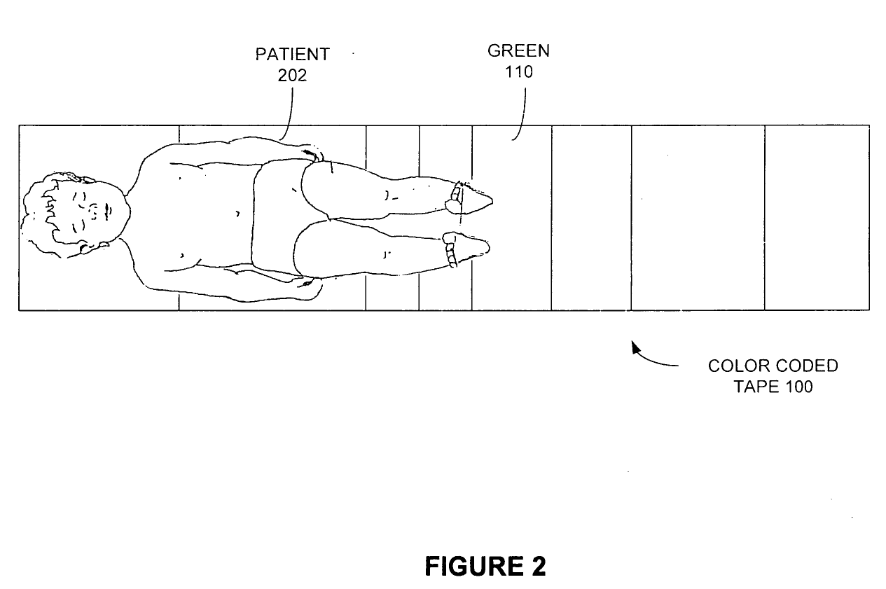

FIG. 2 is an exemplary view of a patient 202 measured head to feet by the color coded tape 100 of FIG. 1A, according to one embodiment. Once the care provider comes to the aid of the patient 202 (e.g., a child), the patient 202 is first placed on the color coded tape 100. Then, the color matching the category of the patient 202 is determined based on the location of the top of patients head. As illustrated in FIG. 1B, the patient 202 weighing between 15 and 18 kilograms is assigned to the green 110 category.

The category may be further divided into subcategories based on the weight of the patient 202. For example, the treatment category of the green 110 which ranges between 15 to 18 kilograms may be further divided into two subcategories, the first subcategory ranging between 15 and 17 kilograms, and the second subcategory ranging between 17 and 18 kilograms. To treat the patient 202, the care provider may carry pre-hospital pediatric medical equipments, drugs, and their instructions predetermined for this category (e.g., the green 110).

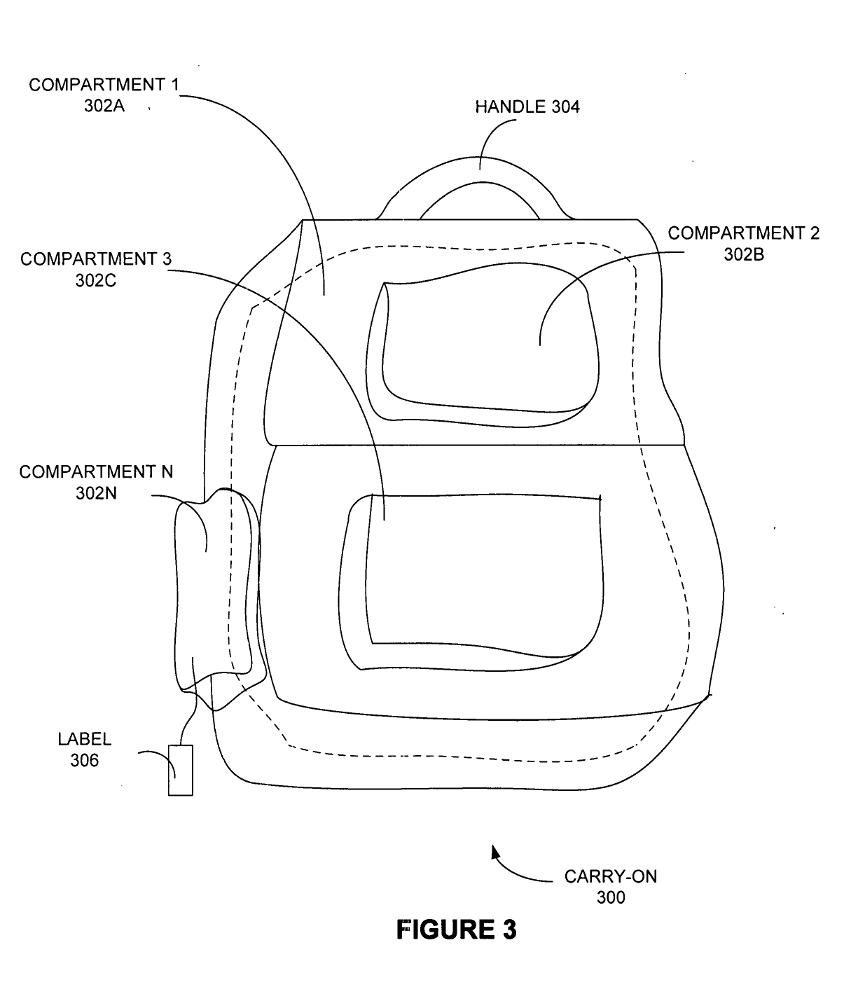

FIG. 3 is an exemplary view of a carry-on with multiple compartments, according to one embodiment as may be brought by the care provider along with the measurement tape of FIG. 1A in response to an emergency call. The pre-hospital pediatric medical treatment often takes place outside the realm of a hospital. Thus, the care provider is often placed into the situation of carrying medical equipment, drugs, and/or instructions (e.g., medical procedures, dosages, etc.). Since the care provider is often on the road, it is ideal to have a light weight and organized care package. In one example embodiment, a portable container (e.g., a carry-on) may be used to carry items (e.g., the care package such as medical equipments, drugs, etc.). The portable container could have multiple compartments (e.g., a compartment 1 302A, a compartment 2 302B, a compartment 3 302C, and/or a compartment N 302N) as illustrated in FIG. 3. The compartments may be closed using zippers and the zippers may have tags indicating the contents of the compartments for quick item location.

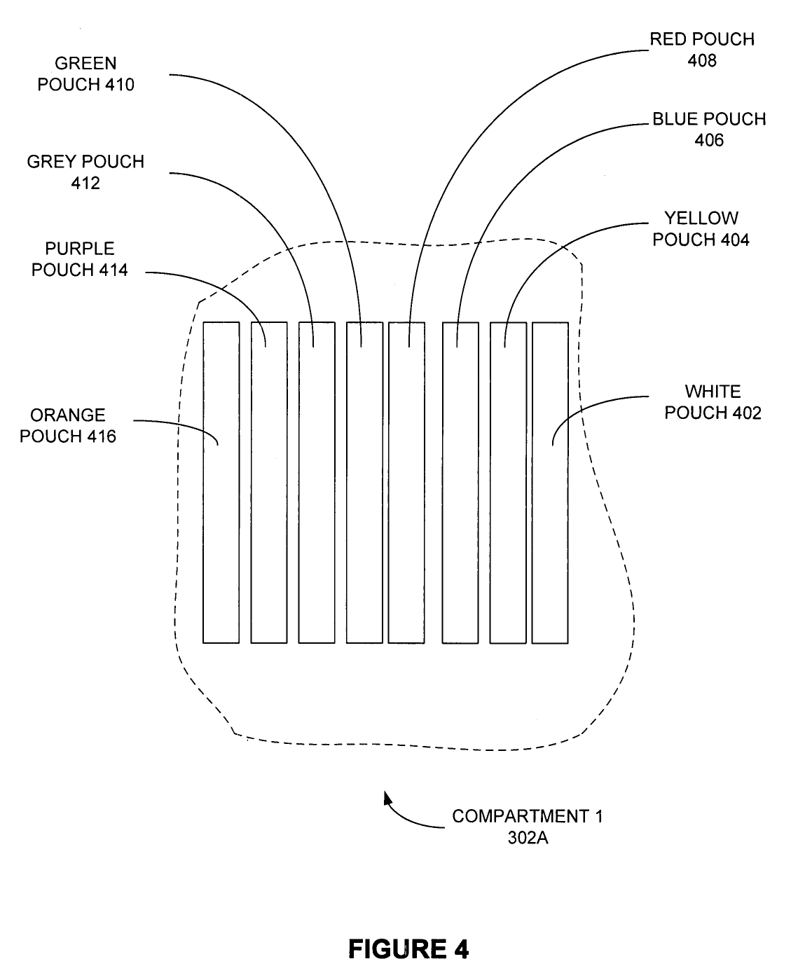

The compartment 1 302A (e.g., the main compartment) contains multiple color-coded pouches, as will be illustrated in more details in FIG. 4. The compartment 2 302B, the compartment 3 302C, and the compartment N 302N may contain medical equipment commonly used for the patient regardless of the patient's size, such as the color-code tape 100, an infant and/or child bag valve mask (BVM) and/or blood pressure (BP) cuff, a stethoscope, an obstetrics (OB) kit, an intravenous (IV) start kit, a laryngoscope handle, a McGill forceps, as mall endotracheal tube holder, a tidal CO2 detector, a King Tube, and/or an inflation syringe. In another example embodiment, a label (e.g., a metal tag) may be attached to each of the multiple compartments to indicate the contents of the compartments.

FIG. 4 is an exemplary view of a main treatment compartment of the carry-on of FIG. 3 having multiples pouches in different colors, according to one embodiment. The main compartment (e.g., the compartment 1 302A) contains a white pouch 402, a yellow pouch 404, a blue pouch 406, a red pouch 408, a green pouch 410, a grey pouch 412, a purple pouch 414, and an orange pouch 416. The multiple pouches are painted in different colors to match items in each pouch with the categories (e.g., as illustrated in the category table 150 of FIG. 1) available for the patient.

For example, the white pouch 402 is used to treat a neonatal patient in an urgent medical crisis, the yellow pouch 404 to treat a patient weighing between 3 to 9 kilograms, the blue pouch 406 to treat a patient weighing between 9 to 11 kilograms, the red pouch 408 to treat a patient weighing between 11 to 15 kilograms, the green pouch to treat a patient weighing between 15 to 18 kilograms, the grey pouch to treat a patient weighing between 18 to 22 kilograms, the purple pouch to treat a patient weighing between 22 to 30 kilograms, and the orange pouch to treat a patient weighing between 30 to 36 kilograms. Additionally, another colored pouch may be added to the main compartment to treat a patient with a prenatal emergency.

The items in the colored pouches may include medical equipment, medications, and color-coded pocket sized books listing symptoms, packing lists, and medical procedures. The medical equipments and medications in a colored pouch are used to treat the patient who is assigned to use the colored pouch. The equipment may be sized approximately for the weight class of the patient. The associated treatment index card may have medicine dosages that are appropriate for the patient weight class. A tape measure (e.g., approximating the weight of the patient from his or her height) may be used to determine the category (e.g., associated with certain color) of the patient.

The color-coded pocket sized books (e.g., multiple pages of reference cards), or treatment index or reference cards share same color as the colored pouches. Alternatively, the color-coded pocket sized books may be combined into a single emergency manual with multiple colored sections (e.g., with each section representing a different patient category). The color-coded pocket sized books or the emergency manual may be prepared according to the protocol mandated by a local government (e.g., a state, a county, a city, a municipality, etc.) and/or include medicine dosages and procedures specific to the color coded weight class to which they belong. These colored books include symptom specific dosages and treatment protocols applicable for their weight classes.

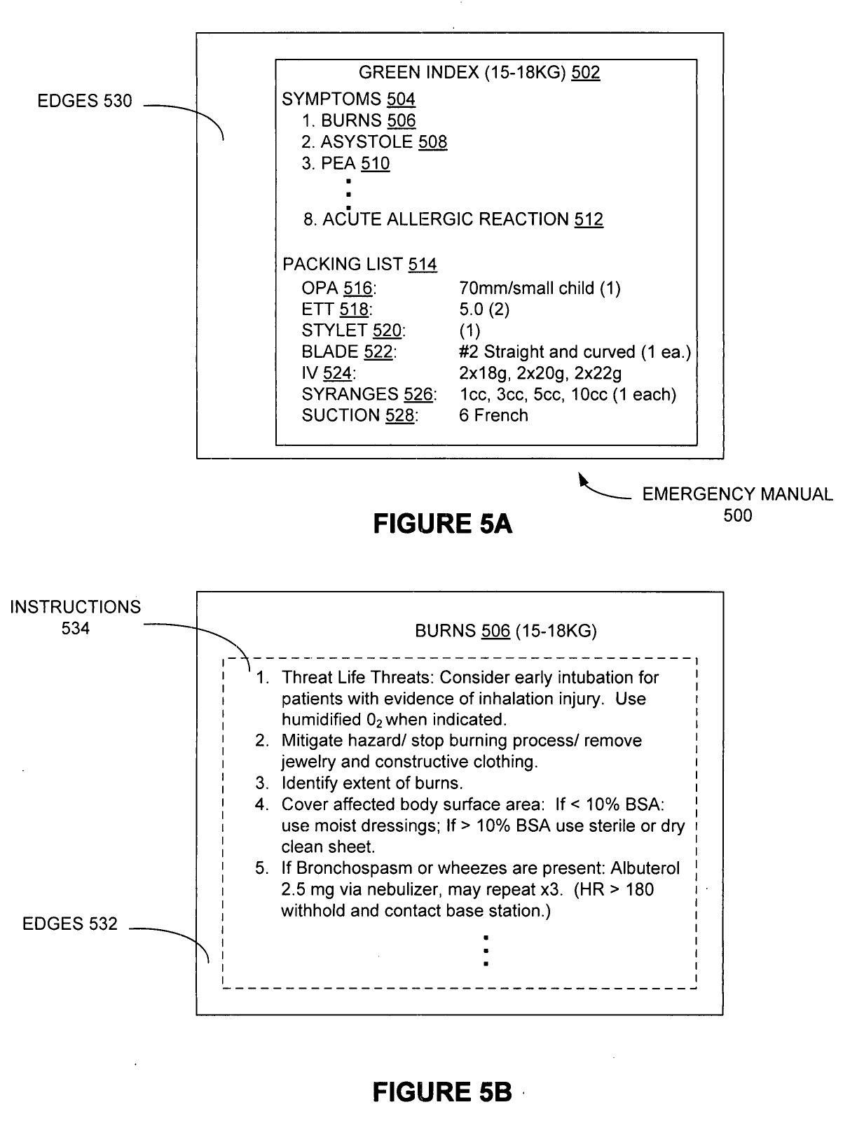

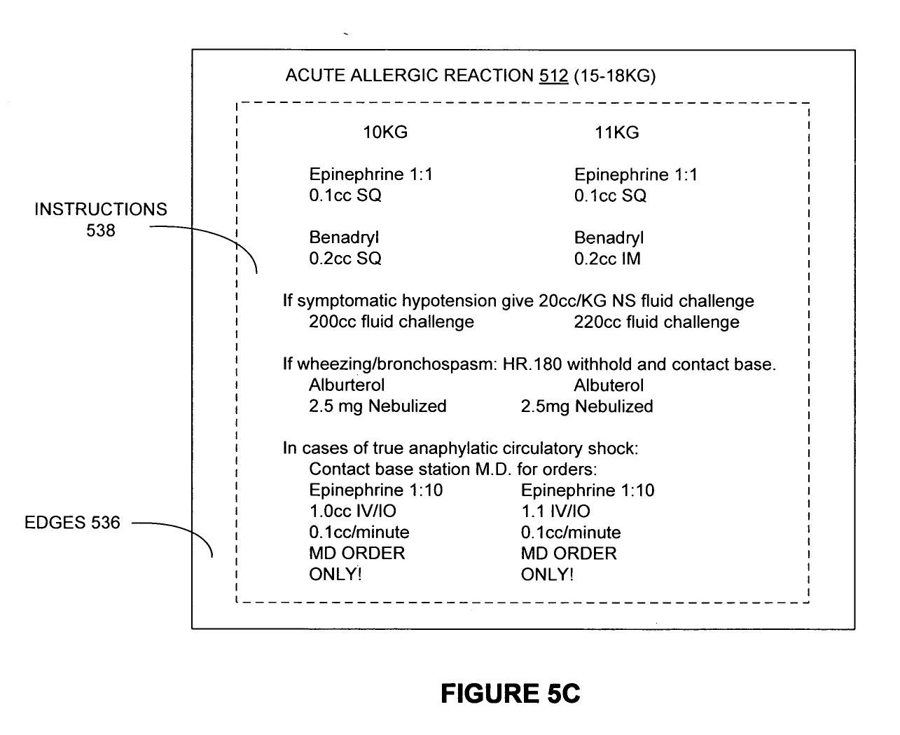

FIG. 5A is an exemplary view of an emergency manual 500 listing symptoms and a packing list, according to one embodiment. The emergency manual 500 may be color-coded pocket sized reference cards (e.g., to easily fit into the colored pouches of FIG. 4) or a single booklet. FIG. 5A illustrates the emergency manual 500 in the reference card format. The green index 502 indicates that information (e.g., dosages, treatment protocol, etc.) listed on the reference cards is geared toward treating a patient with his or her weight ranging from 15 to 18 kilograms. The edges 530 of the reference cards are painted in green to easily distinguish the category of the patient covered by the emergency manual 500.

The green index 503 lists symptoms 504 (e.g., burns 506, asystole 508, pea 510, acute allergic reaction 512, etc.) as well as a packing list 514 (e.g., an oropharyngeal airway (OPA) 516, an endotracheal tube (ETT) 518, a stylet 520, stomatostyle (STYLET) 520, a blade 522, an intravenous (IV) 524, syringes 526, and a suction 528). The packing list 514 describes what is needed to be included in the green pouch along with the reference cards.

FIG. 5B is an exemplary page view of the emergency manual of FIG. 5A listing instructions 534, according to one embodiment. In FIG. 5B, the reference cards list instructions to treat a patient with the burns 506. The instructions 534 are a set of medical procedures that need to be carried out by a care giver. The instructions 534 also list the medical equipment and/or dosages to treat the burns 506. The edges 532 of the reference cards are colored green for the prompt recognition of the patient category. It is appreciated that the books and included treatment protocol and dosages are all specific to the geographic location in which the emergency occurs.

FIG. 5C is an exemplary page view of the emergency manual of FIG. 5A listing instructions 538, according to one embodiment. In FIG. 5C, the reference cards list instructions to treat a patient with the acute allergic reaction 512. The instructions 538 are a set of medical procedures that need to be carried out by a care giver. The instructions 538 also list the medical equipment and/or dosages to treat the symptom.

As illustrated in FIG. 5C, the dosages for specific symptoms are listed according to subcategories of the weight class. In addition, the instruction makes it simpler for the care giver to follow the dosages or instructions mandated by the governing body of the locality. For example, the unit of the dosages on the instructions 538 may be converted to a unit familiar to the care giver, so any mistake due to misunderstanding of the dosage unit may be minimized. Furthermore, the instructions 538 are written in a larger font and/or simplified manner such that the care giver does not have to reread to understand the content.

The edges 536 of the reference cards are colored green for the prompt recognition of the patient category. It is appreciated that the books and included treatment protocol and dosages are all specific to the geographic location in which the emergency occurs.

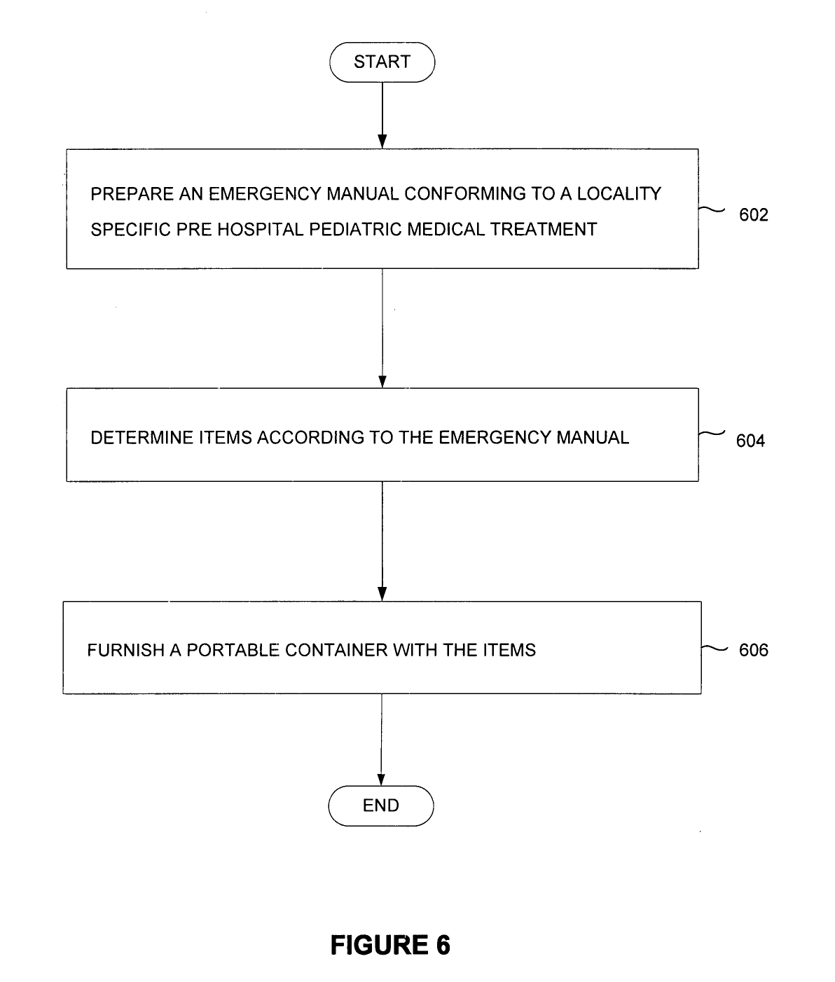

FIG. 6 is an exemplary process flow chart of furnishing a portable container based on a locality specific pre-hospital pediatric medical treatment guideline, according to one embodiment. In operation 602, an emergency manual conforming to the locality specific pre-hospital pediatric medical treatment (e.g., which may be mandated by a county, a city, a municipality, etc.) may be prepared.

In operation 604, items (e.g., a measuring tape, medical equipments, etc.) emergency manual are determined according to the emergency manual. In operation 606, a portable container (e.g., a carry-on) is furnished with the items. The portable container may include a number of pouches in multiple colors with each pouch containing some of the items.

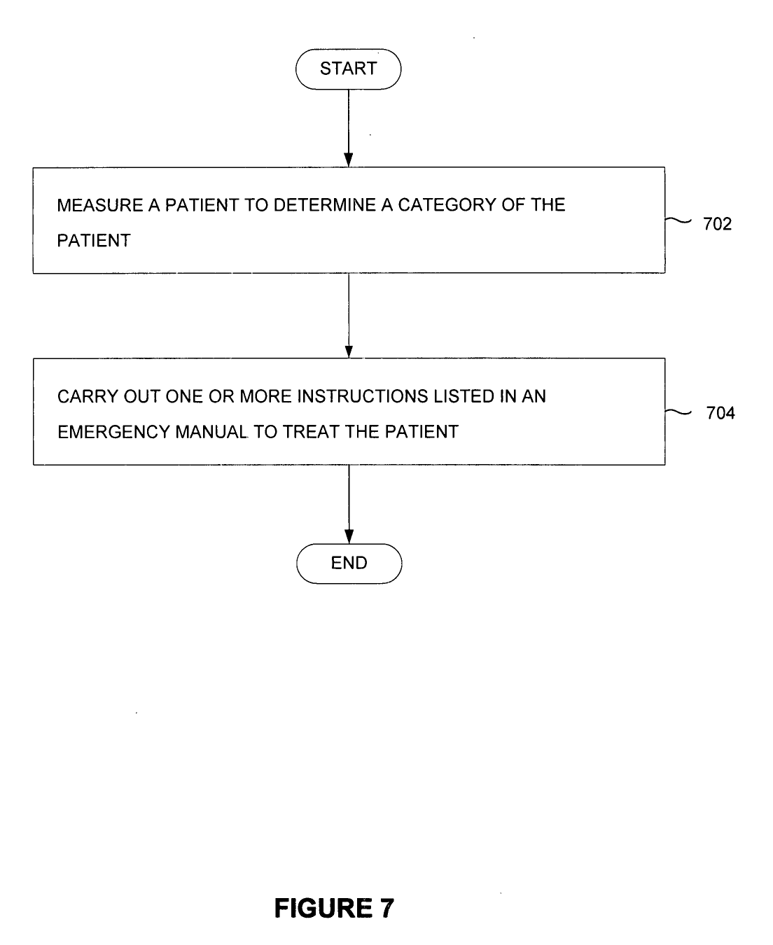

FIG. 7 is an exemplary process flow chart of administering a locality specific pre-hospital pediatric medical treatment, according to one embodiment. In operation 702, a patient is measured to determine a category of the patient. In operation 704, one or more instructions (e.g., medical procedures) listed in an emergency manual are carried out to treat the patient (e.g., to provide an optimal airway access, an optimal intravenous access, and an optimal administration of medication to the patient). Additionally, a pouch containing items necessary for the medical procedures (e.g., medical equipments, medications, etc.) may be selected.

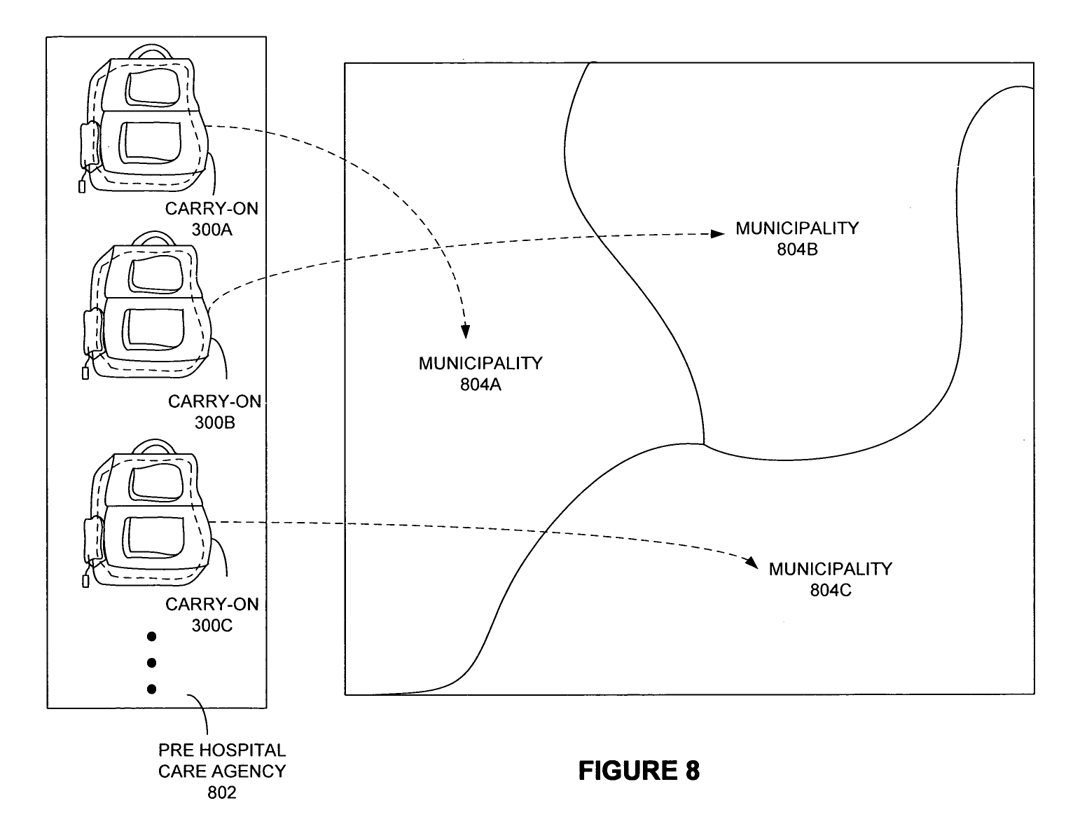

FIG. 8 is an exemplary view of a pre-hospital emergency care agency 802 serving emergency needs of three different municipalities by methods and tools described in FIGS. 1A through 7, according to one embodiment. As illustrated in FIG. 8, the pre-hospital emergency care agency 802 may be contracted to serve the emergency needs of a locality A 804, a locality B 806, and a locality C 808.

The localities may be a municipality, a city, a county, and/or a state. In one example embodiment, the localities may have different pre-hospital medical treatment protocols (e.g., pediatric). Thus, the pre-hospital emergency care workers who work for the pre-hospital emergency care agency 802 may be required to follow three different emergency protocols mandated by the three different localities.

In order to help the emergency care workers to execute the different protocols promptly and/or precisely, different emergency care packages (e.g., a carry-on 300A, a carry-on 300B, a carry-on 300C, etc.) may be prepared to serve individual needs of the three different localities. For instance, the carry-on 300A may be equipped with a measuring tape (e.g., which is divided into multiple sections matching patient classes), an emergency manual (e.g., in colored index cards with each color matching one of the patient classes), several colored pouches with each color matching one of the patient classes, and equipment and drugs common to every patient. Each of the colored pouches may include equipment and drugs unique to the patient class associated with the pouch.

When an emergency care worker (e.g., a paramedic) is called to treat a patient who is residing in the locality A 804, the emergency care worker may take the carry-on 300A. Once the emergency care worker arrives to the scene, the worker first may measure the height of the patient to find out about the weight class of the patient. With that information, the emergency care worker may promptly locate a particular pouch associated with the weight class helped by the color of the pouch (e.g., which shares the same color as the portion of the measuring tape indicating the weight class of the patient).

Once the pouch is located, the emergency care worker may administer the patient with the equipment and drugs contained in the pouch. Additionally, pages of colored index cards (e.g., which may be bound in rings) may describe the medical procedures and dosages associated with symptoms and the weight class of the patient. The emergency care worker may take time to read the index cards in details if he or she is not familiar with the locality A 804. Because the index cards may be prepared in large letters containing essential information, the detailed reading may be brief. The index cards may also be useful to remind the emergency care worker the exact amounts of the dosages. Furthermore, one or more compartments of the carry-on 300A may include equipment and medications common to different weight classes of the patient.

This way, more precise and prompt administration of the pre-hospital emergency care may be administered while single pre-hospital emergency care agency 802 serving a single locality or multiple localities.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. An apparatus for medical treatment, comprising:
a measuring tape divided into multiple sections with each section representing a different category of a patient;
an emergency manual based on a locality specific pre-hospital pediatric medical treatment listing information for treating the patient; and
a plurality of pouches containing items mandated by the emergency manual, wherein the plurality of pouches correspond to the multiple sections.

2. The apparatus of claim 1, wherein the locality specific pre-hospital pediatric medical treatment is mandated by at least one of: a state; a county; a city; and a municipality.

3. The apparatus of claim 1, wherein the measuring tape comprises different colors to distinguish the multiple sections.

4. The apparatus of claim 1, wherein the measuring tape is made of a non-absorbing material which adheres to a surface disposed under the measuring tape without the measuring tape curling up.

5. The apparatus of claim 1, wherein the items comprise section specific medical equipment and medication.

6. The apparatus of claim 1, wherein the plurality of pouches are color coded to match colors assigned to the multiple sections on the measuring tape.

7. The apparatus of claim 1, wherein the emergency manual lists symptoms, a packing list, and instructions for each of the multiple sections.

8. The apparatus of claim 1, wherein the emergency manual comprises a set of color coded pocket sized books with each color coded pocket sized book corresponding to one of the multiple sections.

9. The apparatus of claim 1, wherein the plurality of pouches comprises colored index cards with each color of the plurality of pouches matching each color of the index cards.

10. The apparatus of claim 1, further comprising a carry-on having multiple compartments, wherein the plurality of pouches is contained in one of the multiple compartments.

11. The apparatus of claim 10, wherein the multiple compartments comprise a compartment containing medical equipment common to treating a patient irrespective of the different category of the patient.

12. The apparatus of claim 9, further comprising a respective label tagged to each of the multiple compartments indicating contents of the multiple compartments.

13. A method for pre-hospital pediatric medical treatment, comprising:
preparing an emergency manual conforming to a locality specific pre hospital pediatric medical treatment,
wherein the emergency manual comprises a plurality of sections divided according to a weight class of a patient;
determining items according to the emergency manual for each of the weight class; and
furnishing a portable container with the items.

14. The method of claim 13, wherein the furnishing the portable container comprises equipping some of the items in a plurality of pouches, wherein each of the plurality of pouches corresponds to a different weight class.

15. The method of claim 14, further comprising coloring the plurality of pouches with different colors with each color corresponding to the different weight class.

16. The method of claim 13, wherein the furnishing the portable container comprises equipping the portable container with a measuring tape divided into the plurality of sections.

17. The method of claim 13, wherein the items comprise medical equipment to treat a patient with a prenatal or neonatal emergency.

18. A method for pre-hospital pediatric medical treatment, comprising:
measuring a patient to determine a weight category of the patient;
locating equipment and information specific to the weight category of the patient, wherein the information is located in an emergency booklet; and
carrying out at least one instruction listed in the emergency booklet to treat the patient,
wherein the emergency booklet conforms to a locality specific pre-hospital pediatric medical treatment and lists medical procedures for treating the category of the patient.

19. The method of claim 18, wherein the locating equipment comprises selecting a pouch containing items necessary to perform the medical procedures for treating the category of the patient.

20. The method of claim 18, wherein the measuring the patient comprises determining the weight category of the patient by using a color-coded height-to-weight conversion tape measure.

21. The method of claim 18, wherein the carrying out the at least one instruction comprises providing at least one of an optimal airway access, an optimal intravenous access, and an optimal administration to the patient.
